(19) United States
(12) Patent Application Publication
(10) Pub. No.: US 2021/0056159 A1
(43) Pub. Date: Feb. 25, 2021

(54) CALCULATION OF INTERMOLECULAR FORCES FOR THE DESIGN OF PHYSICAL AND MECHANICAL PARAMETERS

(71) Applicants: Anna Sorokina, Boston, MA (US); Valeriy Anisimov, Syzran (RU)

(72) Inventors: Anna Sorokina, Boston, MA (US); Valeriy Anisimov, Syzran (RU)

(21) Appl. No.: 16/545,099

(22) Filed: Aug. 20, 2019

(51) Int. Cl. G06F 17/11; C23C 14/34; C23C 14/54; G16C 10/00; G06F 16/25

(52) U.S. Cl. CPC G06F 17/11; C23C 14/34; G06F 16/252; G16C 10/00; C23C 14/54

(57) ABSTRACT

A system for calculating intermolecular forces over a communications network includes a computing device including a processor, a memory, an attached database, a user interface, a display and a programming module configured for reading initial data input by a user reading physical constants corresponding to a specific molecule, calculating a non-linear relationship between stresses and deformation of a comprehensive tension-compression of the specific molecule, an energy of sublimation of the specific molecule, parameters of the specific molecule, an interaction force between the specific molecule and an external surface of its body, a force acting on the specific molecule, wherein its displacement is relative to other molecules, and transmitting said resulting data. The system also includes a physical vapor deposition vacuum process system used to deposit a very thin film onto a substrate, which system adjusts a voltage according to data from the computing device.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The claimed subject matter relates to the field of chemistry and, more specifically, the claimed subject matter relates to the field of intermolecular forces that mediate interactions between molecules.

### BACKGROUND

Intermolecular forces are the forces which mediate interaction between molecules, including forces of attraction or repulsion that act between molecules and other types of neighboring particles, e.g., atoms or ions. Intermolecular forces are an essential part of force fields frequently used in molecular mechanics. Molecular interactions are important in diverse fields of protein folding, drug design, material science, sensors, nanotechnology, separations, and origins of life. Molecular interactions are also known as noncovalent interactions or intermolecular interactions.

Various formulas are available for the study of such interactions of molecules. Known mechanical constants (e.g., elastic modulus, bulk modulus, Poisson's ratio) and physical constants (e.g., heat capacity, thermal expansion coefficient, sublimation energy, heat of fusion, volume change during melting) are used to calculate the parameters of molecular interaction. But the currently available formulas for calculating the parameters of molecular interaction utilize functions of, or intermolecular potential of, molecules. Information on the potential of molecules is based on the data about various macroscopic properties of a substance. Functions are based on measuring the deviation of the corresponding value of the property of a real substance with respect to the same property, but related to the ideal state of the substance. Thermophysical quantities can be taken as measured values, such as viscosity, diffusion, particle distance parameters, etc. A function is the relationship between the energy of molecular interactions and the distance between the molecules. However, the potential obtained from the data of one property may differ significantly from the potential obtained from the data of another property. Therefore, to determine the correct potential, a time-consuming procedure is required to optimize the largest possible number of experimental data. At the same time, the reliability of the information received inevitably decreases.

In another approach for the study of such interactions of molecules, a triple integral in spherical coordinates is used to describe the rheological state of materials. The disadvantage of the method of describing the rheological state of materials in the monograph is that it is of a purely modeled (non-practical) nature. The main drawback of the conventional approaches above for the study of such interactions of molecules is that only physical constants (viscosity, diffusion, particle distance parameters, etc.) are used to find the interaction forces between the molecules. This is lacking in situations where precise calculations of intermolecular forces are needed for the physical and mechanical parameters in industrial processes, such as physical vapor deposition vacuum process systems and high-pressure, high-temperature press systems.

Therefore, what is needed is a system and method for improving the problems with the prior art, and more particularly for a more expedient and efficient method and system for improving the accuracy of calculations of intermolecular forces.

### BRIEF SUMMARY

In one embodiment, a system for calculating intermolecular forces over a communications network is disclosed. The system includes a computing device communicably connected to a communications network, the computing device including a processor, a memory, an attached database, a user interface, a display and a programming module configured for: 1) reading initial data input by a user into the user interface, wherein said initial data includes an identity of a specific molecule, 2) reading physical constants corresponding to the specific molecule from the database, wherein said physical constants include elastic modulus, bulk elastic modulus, Poisson's ratio, heat capacity, thermal expansion coefficient, sublimation energy, heat of fusion, and volume change during melting, 3) calculating the following resulting data based on said initial data and said physical constraints: i) a non-linear relationship between stresses and deformation of a comprehensive tension-compression of the specific molecule, ii) an energy of sublimation of the specific molecule, iii) parameters

$$ξ = \frac{r}{{r}_{o}} ; α = \frac{{r}_{1}}{{r}_{n}} ; β = {br}_{0} ; {A}_{0} = \frac{A}{{r}_{o}}$$

of the specific molecule, iv) an interaction force between the specific molecule and an external surface of its body, v) a force acting on the specific molecule, wherein its displacement is relative to other molecules, and 4) transmitting said resulting data to an exterior node, over the communications network, The system also includes a physical vapor deposition vacuum process system used to deposit a very thin film onto a substrate, the system including a computer communicably connected to the communications network, the computer including a processor, a memory, and a programming module configured for: I) receiving said resulting data from the computing device, over the communications network, and 2) adjusting a voltage, and a time of application of said voltage, in the physical vapor deposition vacuum process system according to said resulting data.

Additional aspects of the claimed subject matter will be set forth in part in the description which follows, and in part will be obvious from the description, or may be learned by practice of the claimed subject matter. The aspects of the claimed subject matter will be realized and attained by means of the elements and combinations particularly pointed out in the appended claims. It is to be understood that both the foregoing general description and the following detailed description are exemplary and explanatory only and are not restrictive of the disclosed subject matter, as claimed.

### BRIEF DESCRIPTION OF THE SEVERAL VIEWS OF THE DRAWINGS

The accompanying drawings, which are incorporated in and constitute part of this specification, illustrate embodiments of the claimed subject matter and together with the description, serve to explain the principles of the claimed subject matter. The embodiments illustrated herein are presently preferred, it being understood, however, that the claimed subject matter is not limited to the precise arrangements and instrumentalities shown, wherein:

FIG. 1 is a block diagram illustrating the network architecture of a system for calculating intermolecular forces over a communications network, in accordance with one embodiment.

FIG. 2 is a block diagram showing the data flow of the process for calculating intermolecular forces over a communications network, according to one embodiment.

FIG. 3 is a flow chart depicting the general control flow of a process for calculating intermolecular forces over a communications network, according to one embodiment.

FIG. 4 is a block diagram depicting a system including an example computing device and other computing devices.

FIG. 5 is a table of values.

FIG. 6 is a table of values.

FIG. 7 is a table of values.

FIG. 8 is a table of values.

FIG. 9 is a graph.

FIG. 10 is a graph.

FIG. 11 is a graph.

### DETAILED DESCRIPTION

The disclosed embodiments improve upon the problems with the prior art by providing a system and method that improves the accuracy of calculations of intermolecular forces. The claimed methods fill the gap in the current study of parameters of molecule interactions in solid and liquid bodies. The process of the claimed subject matter determines the forces acting on a specified molecule after external exposure, for example, during thermal motion. The process of the claimed subject matter also calculates the energy necessary for the displacement of said specified molecule by such a distance that the molecule moves to a new equilibrium position. The process of the claimed subject matter provides a more accurate and precise description of diffusion of said specified molecule in solids and liquids, properties of multilayer coatings (adhesion between layers), rheological and strength properties of solids, and dependence of the properties of substances on temperature. The process of the claimed subject matter also defines the non-linear relationship between stresses and deformations of all-round tension-compression. The results provided by the process of the claimed subject matter can be used to improve the technology of allotropic modifications of substances (graphite-diamond, amorphous-crystalline silicon, etc.), as well as to create technologies for processing materials with pressure, etc. Knowledge of the interaction forces between molecules allows one to find the criteria that determines the internal state of bodies. The results provided by the process of the claimed subject matter can be used, among other things, to improve deposition technology. In existing sputtering technologies, the force acting between the external molecule and the body is not currently taken into account. The results provided by the process of the claimed subject matter can be used by deposition technology to take the force acting between the external molecule and the body into account.

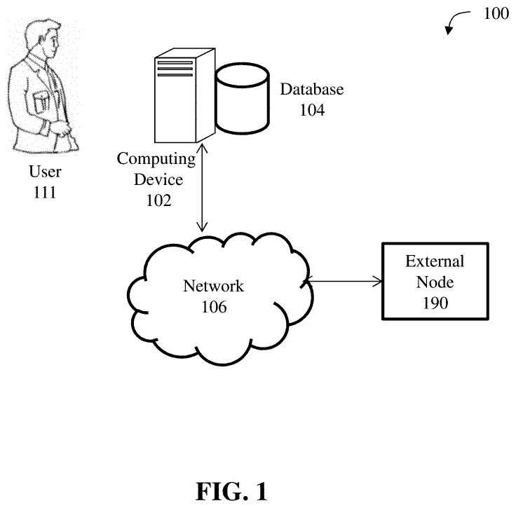

Referring now to the drawing figures in which like reference designators refer to like elements, there is shown in FIG. 1 an illustration of a block diagram showing the network architecture of a system 100 and method for calculating intermolecular forces over a communications network in accordance with one embodiment. A prominent element of FIG. 1 is the computing device 102 associated with repository or database 104 and further communicatively coupled with network 106, which can be a circuit switched network, such as the Public Service Telephone Network (PSTN), or a packet switched network, such as the Internet or the World Wide Web, the global telephone network, a cellular network, a mobile communications network, or any combination of the above. Computing device 102 is a central controller or operator for functionality of the disclosed embodiments, namely, facilitating molecular force calculating activities.

FIG. 1 includes a computing device 102, which may be smart phones, mobile phones, tablet computers, handheld computers, laptops, workstations, desktop computers, servers, laptops, all-in-one computers or the like. In another embodiment, computing devices 102 is AR or VR systems that may include display screens, headsets, heads up displays, helmet mounted display screens, tracking devices, tracking lighthouses or the like. Device 102 may be communicatively coupled with network 106 in a wired or wireless fashion. Augmented reality (AR) adds digital elements to a live view often by using a camera on a computing device. Virtual reality (VR) is a complete or near complete immersion experience that replaces the physical world.

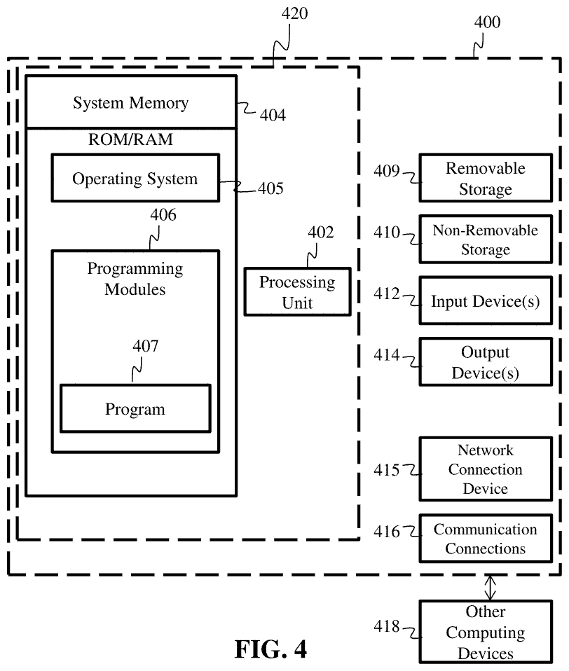

The computing device 102 includes a processor, a memory, a user interface (such as a keyboard, touch screen, mouse, etc.), a display and a programming module, as described more fully below with reference to FIG. 4. FIG. 1 further shows that computing device 102 includes a database or repository 104, which may be a relational database comprising a Structured Query Language (SQL) database stored in a SQL server. External node 190 may also include its own database. The repository 104 serves data from a database, which is a repository for data used by device 102 and device 190 during the course of operation of the disclosed embodiments. Database 104 may be distributed over one or more nodes or locations that are connected via network 106.

The database 104 may include a record for each specific molecule. A record may include: identifying information for each molecule, a unique identifier for each molecule, elastic modulus, bulk elastic modulus, Poisson's ratio, heat capacity, thermal expansion coefficient, sublimation energy, heat of fusion, and volume change during melting, etc.

FIG. 1 also shows an external node 190, which receives information from computing device 102 and acts accordingly. In one embodiment, the external node 190 is a physical vapor deposition vacuum process system. A physical vapor deposition vacuum process deposits very thin films onto a substrate for a wide variety of commercial and scientific purposes. Sputtering occurs when an ionized gas molecule is used to displace atoms of a specific material. These atoms then bond at the atomic level to a substrate and create a thin film. Several types of sputtering processes exist, including ion beam sputtering, diode sputtering, and magnetron sputtering. In a magnetron sputtering application, the high voltage is delivered across a low-pressure gas (usually argon) in order to create high-energy plasma. These energized plasma ions strike a target composed of the desired coating material. The force causes atoms to eject from the target material and bond with those of the substrate. A physical vapor deposition vacuum process can be used in a variety of industries to create smaller, lighter, more durable products.

In this embodiment, the external node 190 is a physical vapor deposition vacuum process system used to deposit a very thin film onto a substrate, the system including a computer communicably connected to the communications network 106, the computer including a processor, a memory, and a programming module configured for: 1) receiving data from the computing device 102, over the communications network 106, and 2) adjusting a voltage, and a time of application of said voltage, in the physical vapor deposition vacuum process system according to said data.

In another embodiment, the external node 190 is a high-pressure, high-temperature press system that includes a large press that can weigh hundreds of tons and produce a pressure of up to 5 GPa at up to 1500° C. The press supplies the pressure and temperature necessary to produce an allotrope, such as a synthetic diamond. Various presses may be used, such as the belt press, the cubic press and the split-sphere (BARS) press. Seeds of the allotropic element may be placed at the bottom of the press. The internal part of the press is heated above 1400° C. and melts the solvent metal. The molten metal dissolves a high purity source of the allotropic element, which is then transported to seeds and precipitates, forming an allotrope of the allotropic element.

The belt press may include upper and lower anvils that supply the pressure load to a cylindrical inner cell. This internal pressure is confined radially by a belt of pre-stressed steel bands. The anvils also serve as electrodes providing electric current to the compressed cell. A variation of the belt press uses hydraulic pressure, rather than steel belts, to confine the internal pressure. A cubic press includes anvils which provide pressure simultaneously onto all faces of a cube-shaped volume. A cubic press is typically smaller than a belt press and can more rapidly achieve the pressure and temperature necessary to create synthetic diamond. The BARS apparatus includes a ceramic cylindrical synthesis capsule of small size. The cell is placed into a cube of pressure-transmitting material, which is pressed by inner anvils. The outer octahedral cavity is pressed by outer anvils. After mounting, the whole assembly is locked in a disc-type barrel that is filled with oil, which pressurizes upon heating, and the oil pressure is transferred to the central cell. The synthesis capsule is heated up by a coaxial graphite heater and the temperature is measured with a thermocouple.

In this embodiment, the external node 190 is a high-pressure, high-temperature press system used to produce an allotrope of an element, the system including a computer communicably connected to the communications network, the computer including a processor, a memory, and a programming module configured for: 1) receiving said resulting data from the computing device, over the communications network; and 2) adjusting a temperature, pressure, and a time of application of said temperature and pressure, in the high-pressure, high-temperature press system according to said resulting data.

FIG. 1 shows an embodiment wherein networked computing devices 190 interact with device 102 and repository 104 over the network 106. It should be noted that although FIG. 1 shows only the networked computers 190 and 102, the system of the disclosed embodiments supports any number of networked computing devices connected via network 106. Further, device 10, and node 190 include program logic such as computer programs, mobile applications, executable files or computer instructions (including computer source code, scripting language code or interpreted language code that may be compiled to produce an executable file or that may be interpreted at run-time) that perform various functions of the disclosed embodiments.

Note that although computing device 102 and external node 190 are each shown as a single and independent entity, in one embodiment, the functions of computing device 102 and external node 190 may be integrated with another entity. Further, computing device 102 and its functionality, according to a preferred embodiment, can be realized in a centralized fashion in one computer system or in a distributed fashion wherein different elements are spread across several interconnected computer systems.

The computing device 102, via its processor, memory, and attached database, may be configured to perform a variety of calculations, that are described more fully below. The computing device 102 is configured to determine the forces acting on a specified molecule from the molecules surrounding it. As one molecule is released, the surrounding molecules are integrally distributed around it. This is explained by a large number of molecules surrounding the molecule, as well as by their movement. The molecules surrounding a single molecule are considered as a continuous medium.

If we assume that the interaction forces between molecules are radial, then the equilibrium equation for a single molecule, projected on the xᵢ axis, will be as follows:

$${V}_{0} {φ}_{i} ( {x}_{1} , {x}_{2} {x}_{3} ) = \int \int {\int }_{V} F ( r + Δ r ) N ( {x}_{1} + Δ {x}_{1} , {x}_{2} + Δ {x}_{2} , {x}_{3} + Δ {x}_{3} ) \frac{Δ {x}_{i} + Δ {u}_{i}^{*}}{r + Δ r} dV \qquad ( 1 )$$

Description of symbols:

x₁, x₂, x₃—Cartesian coordinates of a single molecule;

Δx₁, Δx₂, Δx₃—Cartesian coordinates of the surrounding molecules, relative to an individual molecule;

V₀—volume of one molecule;

Φᵢ(x₁,x₂,x₃)—the projection on the xᵢ (i=1; 2; 3) of the volume force acting at the point of the molecule;

volumetric force—force reduced to one cubic meter;

F(r)—the force of interaction between two molecules;

Vᵢ— the displacement of a single molecule, for example, due to thermal motion;

uᵢ(x₁,x₂,x₃)—movement of molecules of the body relative to a single molecule as a result of external force (electro-magnetic effect, thermal expansion, force action on the surface of the body);

Δuᵢ=uᵢ(x₁+Δx₁,x₂+x₂,x₃+Δx₃)−uᵢ(x₁,x₂, x₃)—changing the position of the molecules of the body after exposure to external forces;

Δu*ᵢ=Δuᵢ−Vᵢ;

r=\sqrt{Δx₁²+Δx₂²+Δx₃²} is the distance between a single molecule and the surrounding molecules before the forces external to the body;

Δr=\sqrt{(Δx₁+Δu*₁)²+(Δx₂+Δu*₂)²+(Δx₃+Δu*₃)²}−r—change of r after external force action;

N(x₁,x₂,x₃)—is the volume concentration of molecules (the number of molecules in one cubic meter).

The region of integration is the exterior of a sphere of radius r₀ (r₀ is the radius of the molecule). For the convenience of using formula (1), we will apply the integral given below in spherical coordinates.

$$\int \int {\int }_{V} F ( r + Δ r ) N ( {x}_{1} + Δ {x}_{1} , {x}_{2} + Δ {x}_{2} , {x}_{3} + Δ {x}_{3} ) \frac{Δ {x}_{i} + Δ {u}_{i}^{*}}{r + Δ r} dV$$

As a result of applying the formula (1) will change and will have the form (2). Formula (2) will determine the force acting on the molecule after external exposure.

$${φ}_{i} ( {x}_{1} , {x}_{2} , {x}_{3} ) = \frac{N}{{V}_{o}} {\int }_{0}^{2 π} d ϕ {\int }_{0}^{π} d θ {\int }_{{r}_{o}}^{∞} F ( r + Δ r ) \frac{Δ {x}_{i} + Δ {u}_{i}^{*}}{r + Δ r} {r}^{2} sin θ dr \qquad ( 2 )$$

Description of symbols: θ and φ are spherical angles.

Using formulas (1,2), one can find the forces acting on the molecule, for example, during thermal motion. The smaller these forces, the more mobile the molecules. Using the formula (2), it is also possible to find the energy necessary for the displacement of a molecule by such a distance that the molecule moves to a new equilibrium position. When this happens diffusion and plastic effects. Formulas (1,2) allow one to describe diffusion in solids and liquids, properties of multilayer coatings (adhesion between layers), theological and strength properties of solids, and dependence of the properties of substances on temperature.

The computing device 102, via its processor, memory, and attached database, is further configured to calculate the relationship between the stresses σ*ᵢⱼ and displacements Δuᵢ. Take Vᵢ=0. Let σ*ₛₜ denote stresses acting in the direction of the axis along the area perpendicular to the x axis. By decomposing the surface forces in three mutually perpendicular planes, we obtain

$$\frac{{S}_{0}}{N} {σ}_{ij}^{*} = {\int }_{0}^{2 π} d ϕ {\int }_{0}^{π / 2} d θ {\int }_{{r}_{o}}^{∞} \text{(} F ( r + Δ r ) \frac{Δ {x}_{i} + Δ {u}_{i}}{r + Δ r} {n}_{j} {r}^{2} sin θ dr . \qquad ( 3 )$$

Description of symbols:

S₀—the cross-sectional area of the body per one molecule;

$${n}_{j} = \frac{Δ {x}_{j}}{r} ;$$

j=1; 2; 3;

In spherical coordinates: n₁=sin θ cos φ; n₂=sin θ sin φ; n₃=cos θ.

The computing device 102, via its processor, memory, and attached database, is further configured to calculate the forces between molecules. Consider the state of all-round tension-compression (deformations and stresses in all directions are the same). In the case of a state of comprehensive tension-compression, formula (3) takes the form:

$$\frac{{S}_{0}}{N} {σ}^{*} ( e ) = {\int }_{0}^{2 π} d ϕ {\int }_{0}^{π / 2} d θ {\int }_{{r}_{n}}^{∞} \text{(} F ( r ( 1 + e ) ) {n}_{i} {n}_{j} {r}^{2} sin θ d r$$

After integration by dφ and dθ we get:

$$\frac{{S}_{0}}{N} {φ}_{ }^{*} ( e ) = \frac{2 π}{3} {\int }_{{r}_{n}}^{∞} F ( r ( 1 + e ) ) {r}^{2} dr \qquad ( 4 )$$

Description of symbols: e, σ*(e)—deformations and stresses of comprehensive tension-compression.

Formula (4) defines the non-linear relationship between stresses and deformations of all-round tension-compression. This formula can be used to improve the technology of allotropic modifications of substances (graphite-diamonds, amorphous-crystalline silicon, etc.), as well as to create technologies for processing materials with pressure, etc.

Let's change the variable in formula (4):

$$ζ = \frac{r}{{r}_{o}} ( 1 + e )$$

As a result, we get:

$${σ}^{*} ( e ) = \frac{2 π {Nr}_{o}^{3}}{3 {{S}_{0} ( 1 + e )}^{2}} {\int }_{( 1 + e )}^{∞} F ( {r}_{0} ζ ) {ζ}^{2} d ζ \qquad ( 5 )$$

We introduce the function F₀(z):

F₀(z)=∫_{z}^{∞}F(r₀ζ)ζ²dζ. (6)

From expression (5) we get:

$$\begin{matrix}{σ}^{*} ( e ) = \frac{2 π {Nr}_{o}^{3}}{3 {{S}_{0} ( 1 + e )}^{3}} {F}_{0} ( 1 + e ) ; & ( 7 ) \\ {σ}^{{*}^{′}} ( e ) = \frac{2 π {Nr}_{o}^{3}}{3 {{S}_{0} ( 1 + e )}^{3}} ( \frac{- 3}{1 + e} {F}_{0} ( 1 + e ) - F ( {r}_{0} ( 1 + e ) ) . & ( 8 )\end{matrix}$$

Description of symbols: σ*′ is a derivative of σ*(e).

Find the work of external forces as e→∞ (sublimation energy):

$${W}_{s} = 3 {\int }_{0}^{∞} {σ}^{*} ( e ) de = \frac{2 π {Nr}_{0}^{s}}{{S}_{n}} {\int }_{0}^{∞} \frac{{F}_{0} ( 1 + e )}{{( 1 + e )}^{2}} de . \qquad ( 9 )$$

Description of symbols: Wₛ—energy of sublimation.

Expressions (7-9) will be used to find F(r).

In 1818, P. Dulong and A. Petit experimentally established a law according to which the heat capacity of all solids at a sufficiently high temperature is a constant value that does not depend on temperature and is about 3R≅25 J/(moth grad). R is the universal gas constant. In the classical theory of solid state physics, this is explained as the sum of the kinetic energy and the potential energy equal to the kinetic energy. The source of potential energy in classical theories is not completely described. This source of the potential part of the heat capacity is the work of internal compression forces σ*(0), which act continuously inside the bodies, on the deformations of thermal expansion.

From formula (7), it follows that the stresses of comprehensive internal compression σ*(0) constantly act in bodies. Hence the following formula connecting the forces between molecules and physical constants:

$${σ}^{*} ( 0 ) = \frac{2 π {Nr}_{0}^{3}}{3 {S}_{n}} {F}_{0} ( 1 ) = \frac{- C}{3 θ} . \qquad ( 10 )$$

Description of symbols:

C=(Cₚ−Cₖ)—potential component of heat capacity;

Cₚ-volumetric heat capacity (table value);

Cₖ— is the kinetic component of heat capacity;

ϑ—linear thermal expansion coefficient.

Derivative σ*′(0) is equal to:

$${σ}^{{*}^{′}} ( 0 ) = \frac{2 π {Nr}_{o}^{3}}{3 {S}_{n}} ( \frac{- 3}{1 + e} {F}_{0} ( 1 ) - F ( {r}_{0} ) ) = 3 K = \frac{E}{1 - 2 v}$$

Description of symbols:

K—volume modulus of elasticity;

E —modulus of elasticity;

ν—Poisson's ratio.

To find the forces between the molecules we will use the following formula:

$$F ( r ) = A \frac{r - {r}_{2}}{{r}^{3}} {e}^{- br} . \qquad ( 11 )$$

Description of symbols:

r is the distance between molecules;

A, r₁, b—constants.

To find the forces between the molecules, the Van-der-Waltz formula is currently used. The use of formula (11) instead of the Van der Waltz formula gave the best results, since formula (11) allows to take into account the energy of sublimation to find the forces between molecules. Express F(r) in terms of the dimensionless variable

$$ξ = \frac{r}{{r}_{n}} \text{:}$$

$$F ( {r}_{0} ξ ) = {A}_{0} \frac{ξ - α}{{ξ}^{2}} {e}^{- β ξ} , \qquad ( 12 )$$

Description of symbols:

$$ξ = \frac{r}{{r}_{o}} ; α = \frac{{r}_{2}}{{r}_{n}} ; β = {br}_{0} ; {A}_{0} = \frac{A}{{r}_{o}} .$$

The values used to find the forces between molecules are expressed through function (12):

$$\begin{matrix}{F}_{0} ( z ) = \frac{{A}_{0}}{{β}^{2}} = ( β z - αβ + 1 ) {e}^{- β ξ} ; & \\ {σ}^{*} ( 0 ) = \frac{2 π {Nr}_{0}^{3}}{3 {S}_{n}} \frac{{A}_{0}}{{β}^{3}} ( 1 - β ( α - 1 ) ) {e}^{- βξ} = \frac{- 0.5 C}{3 α} ; & ( 13 ) \\ {σ}^{{*}^{′}} ( 0 ) = \frac{2 π {Nr}_{0}^{3}}{3 {S}_{n}} \frac{{A}_{0}}{{β}^{2}} ( ( 3 β + {β}^{2} ) ( α - 1 ) - 3 ) {e}^{- βξ} = 3 K \frac{E}{1 - 2 v} ; & ( 14 ) \\ {W}_{s} = \frac{2 π {Nr}_{0}^{3}}{{s}_{n}} {\int }_{0}^{∞} \frac{{F}_{0} ( 1 + e )}{{( 1 + e )}^{s}} de . & ( 15 )\end{matrix}$$

From expressions (13,14) we get:

$$α - 1 = \frac{3 θ + 1}{{β}^{2} θ + β + 3 θβ} ;$$

$${A}_{0} = \frac{3 {S}_{a} B}{2 π {Nr}_{0}^{3}} ; B = \frac{{β}^{2} {σ}^{*} ( 0 )}{( 1 - β ( α - 1 ) {e}^{- βξ}} ,$$

### Where

$$θ = \frac{- C}{9 θ K} = \frac{1 - 4 v}{1 + v}$$

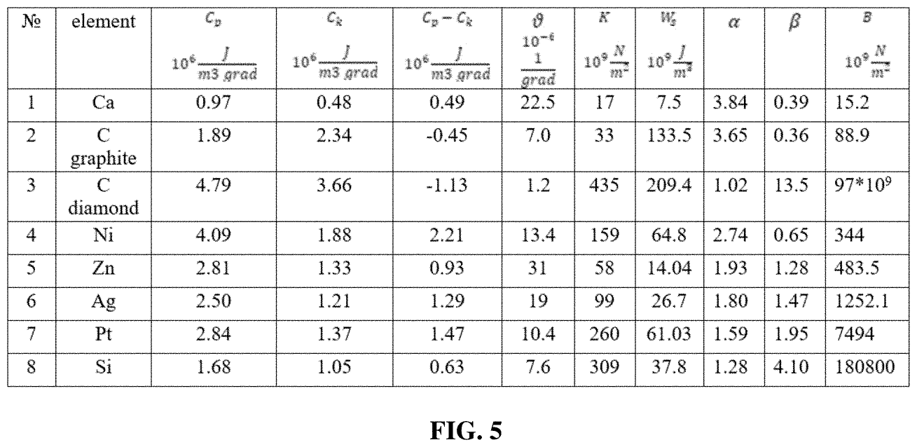

Integral (15) has no analytical solution. The integral was calculated numerically for various values of β. The parameter β was chosen in such a way that equality (15) holds. In the table in FIG. 5, the parameters α, β, B are found for eight substances. The constants A, r₁, b through the table of FIG. 5 α, β, B are found by the following formulas:

$$A = \frac{3 {S}_{0} B}{2 π {Nr}_{0}^{2}} ; {r}_{1} = α {r}_{0} ; b = \frac{β}{{r}_{o}} .$$

Description of symbols in the table of FIG. 5:

Cₚ—volumetric heat capacity;

Cₖ—is the kinetic component of heat capacity;

ϑ—linear thermal expansion coefficient;

K is the bulk modulus;

Wₛ is the energy of sublimation.

These physical quantities are used to find α, β, B. These physical quantities can be found in the attached database 104. The table analysis in FIG. 5 shows as follows. The coefficient B characterizes the hardness of the bodies. The larger the B, the greater the hardness. A diamond has the largest hardness. The parameter

$$α = \frac{{r}_{s}}{{r}_{o}} ,$$

where r₁ is the distance at which the interaction forces between molecules are zero. It characterizes the rarefaction of the substance (the more α, the more sparse the material). For example, for graphite α=3.65, and for diamond α=1.02. The parameter β=br₀ is associated with the energy of sublimation. It characterizes the rate of decrease of intermolecular forces depending on the distance (the smaller the β, the more extended the forces in space). For a covalent bond (for example; Si) β is large (the forces are compressed), and for a metal (for example, Ni) it is small (the forces are stretched). The smaller the β, the individual molecule interacts with a large number of molecules surrounding it.

The computing device 102, via its processor, memory, and attached database, is further configured to calculate the force of interaction between the external molecule and the surface of the body.

$${φ}_{3} ( H ) = \frac{N}{{V}_{o}} \int \int {\int }_{V} F ( r + Δ r ) \frac{{x}_{s} + H}{r + Δ r} dV . \qquad ( 16 )$$

Description of symbols:

H is the distance between the particle and the surface of the body;

x₁, x₂, x₃—Cartesian coordinates of a single body molecule;

r=\sqrt{x₁²+x₂²+x₃²};

r+Δr=\sqrt{x₁²+x₂²+(H+x₃)²}=\sqrt{r²+2Hx₃+H²}.

The area of integration in formula (16) coincides with the area of space occupied by the body. We write the formula (16) in spherical coordinates and introduce dimensionless variables

$$ξ = \frac{r}{{r}_{o}} ; h = \frac{H}{{r}_{o}} .$$

As a result, we get:

$${φ}_{3} ( {r}_{0} h ) = \frac{N}{{V}_{o}} {\int }_{0}^{2 π} d ϕ {\int }_{0}^{π / 2} sin θ d θ {\int }_{{r}_{0}}^{∞} F ( {r}_{0} \sqrt{{ξ}^{2} + 2 h ξ cos θ + {h}^{2}} ) \frac{ξ cos θ + h}{\sqrt{{ξ}^{2} + 2 h ξcos θ + {h}^{2}}} {ξ}^{2} d ξ .$$

After integrating over dφ we get:

$${R}_{3} ( h ) = \frac{{V}_{0}}{2 {S}_{0}} {φ}_{3} ( {r}_{0} h ) = 2 π {\int }_{0}^{π / 2} sin θ d θ {\int }_{{r}_{0}}^{∞} φ ( z ) \frac{ξ cos θ + h}{z} {ξ}^{2} d ξ . \qquad ( 17 )$$

Description of functions:

$$z = \sqrt{{ξ}^{2} + 2 h ξcos θ + {h}^{2}} ; φ ( z ) = \frac{2 π N {r}_{0}^{3}}{3 {S}_{n}} F ( {r}_{0} z ) = B \frac{c - α}{{s}^{2}} {e}^{- {β}_{2}} .$$

Let's find the energy received by the molecule when moving from H₀ [symbol] o H₁:

$$W = {V}_{0} {\int }_{{H}_{0}}^{{H}_{s}} {φ}_{3} ( H ) dH = {r}_{0} {V}_{0} {\int }_{{h}_{0}}^{{h}_{1}} {φ}_{3} ( {r}_{0} h ) dh , \\ r eh = \frac{H}{{r}_{0}} ; {h}_{0} = \frac{{H}_{0}}{{r}_{0}} ; {h}_{1} = \frac{{H}_{1}}{{r}_{0}} . \qquad ( 18 )$$

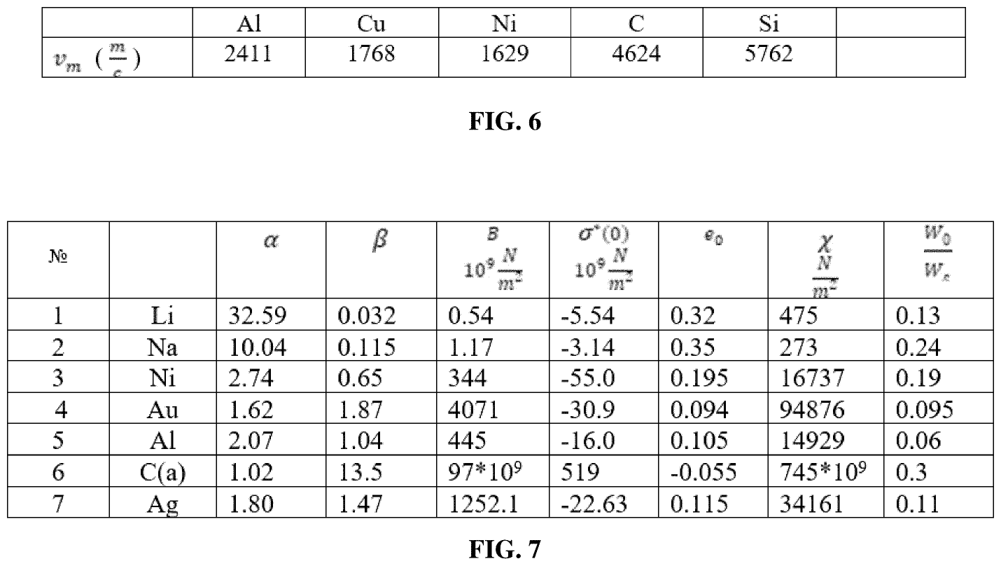

Integrals in expressions (17,18) can be calculated numerically. These formulas can be used, among other things, to improve deposition technology. In existing sputtering technologies, the force acting between the external molecule and the body is not taken into account. The table of FIG. 6 shows the speed vₘ, which is acquired by a molecule that is at infinity with zero speed as it approaches the body. The analysis of the table of FIG. 6 shows that the speeds are high, and they must be taken into account in the deposition technologies.

Knowledge of the interaction forces between molecules allows one to find, among other things, the following criteria that determine the internal state of bodies:

σ*(0) is the internal compression stress, constantly acting in the bodies;

e₀—strain, for which the internal stresses are equal to zero;

W₀ is the hidden energy of internal compression;

χ is the stiffness of the equilibrium state of the molecule (determines the force acting on the molecule, when displaced).

These criteria for the seven substances are given in the table of FIG. 7.

An explanation of the table of FIG. 7 is hereby provided. e₀—characterizes the rarefaction of the molecules of the body. The bigger the value of e₀, the more sparse the bodies. For example, Li is more sparse than Ag. χ—characterizes the mobility of molecules and the hardness of bodies. The smaller the χ, the more mobile the molecules. The more χ, the greater the body hardness.

$$\frac{{W}_{o}}{{W}_{s}}$$

—characterizes the explosiveness of substances. The more

$$\frac{{W}_{o}}{{W}_{s}} ,$$

the greater the risk of an explosion. For example, Li-based batteries should be safer than Na-based batteries. The internal compressive stresses permanently acting in the bodies at the melting temperature can be found by the formula:

$${σ}_{0} = \frac{{L}_{m}}{Δ {V}_{m}}$$

Description of symbols:

Lₘ—is the volume heat of fusion (the amount of heat needed to melt one cubic meter of substance);

ΔVₘ is the relative change in volume during melting.

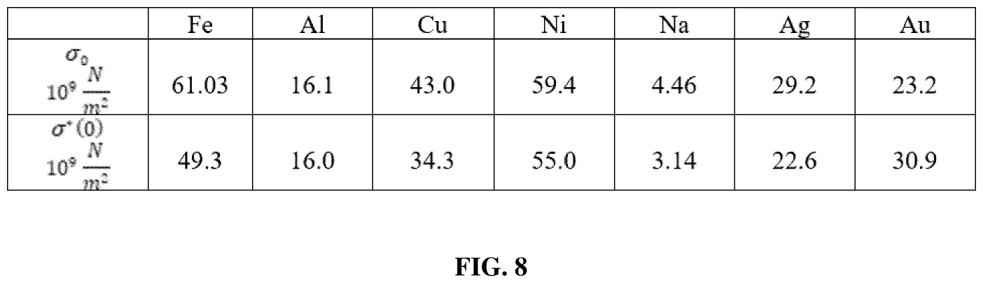

For the table of FIG. 8, σ₀ and σ*(0) are given for seven substances.

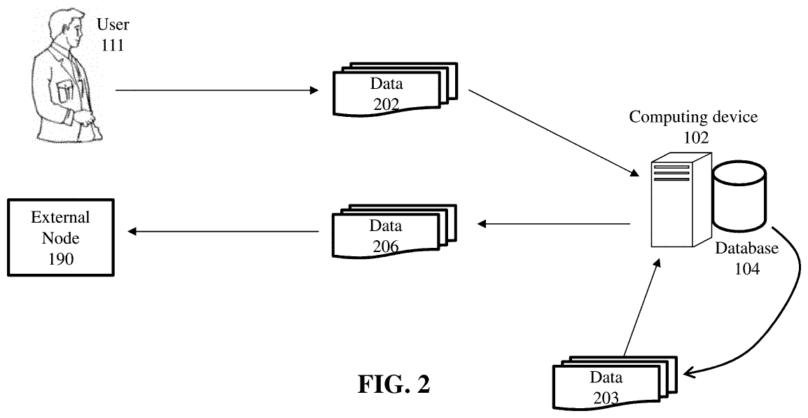

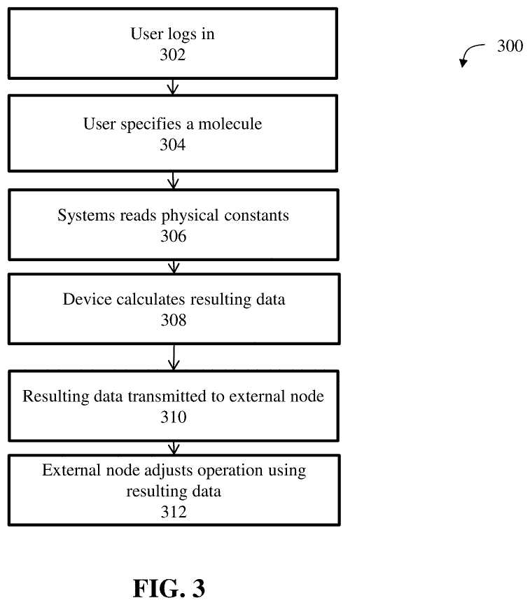

The process of calculating intermolecular forces over a communications network will now be described with reference to FIGS. 2-4 below. FIG. 2 depicts the data flow and control flow of the process for calculating intermolecular forces over a communications network 106, according to one embodiment. The process of the disclosed embodiments begins with optional step 302 (see flowchart 300), wherein the user 111 may log in, enroll or register with computing device 102. In the course of logging in, enrolling or registering, the user 111 may enter data into the computing device 102 by manually entering data into an application via keypad, touchpad, or via voice. In the course of logging in, enrolling or registering, the user 111 may enter any data that may be stored in a record, as defined above.

Subsequently, in step 304, the user 111 may specify a molecule by inputting said data 202 into computing device 102. In the course of inputting said data 202, the user 111 may enter data into the device by manually entering data into an application via keypad, touchpad, or via voice. In the course of entering said data 202, the user may enter any data that may be stored in a record, as defined above.

In step 306, the processor of computing device 102 reads physical constants (data 203) corresponding to the specific molecule from the database 104, wherein said physical constants include elastic modulus, bulk elastic modulus, Poisson's ratio, heat capacity, thermal expansion coefficient, sublimation energy, heat of fusion, and volume change during melting.

Next, in step 308, the processor of computing device 102 calculates the following resulting data 206 based on said initial data and said physical constraints. The processor of computing device 102 first calculates a non-linear relationship between stresses and deformation of a comprehensive tension-compression of the specific molecule using Formula (4) above. Then, the processor of computing device 102 calculates an energy of sublimation of the specific molecule using Formula (9) above. Next, the processor of computing device 102 calculates the parameters

$$ξ = \frac{r}{{r}_{0}} ; α = \frac{{r}_{2}}{{r}_{0}} ; β = {br}_{0} ; {A}_{0} = \frac{A}{{r}_{0}}$$

of Formula (12) above of the specific molecule. The processor of computing device 102 performs this step using Formulas (13), (14) and (15) above. Subsequently, the processor of computing device 102 calculates an interaction force between the specific molecule and an external surface of its body using Formula (17) above. Finally, the processor of computing device 102 calculates a force acting on the specific molecule, wherein its displacement is relative to other molecules using Formula (2) above.

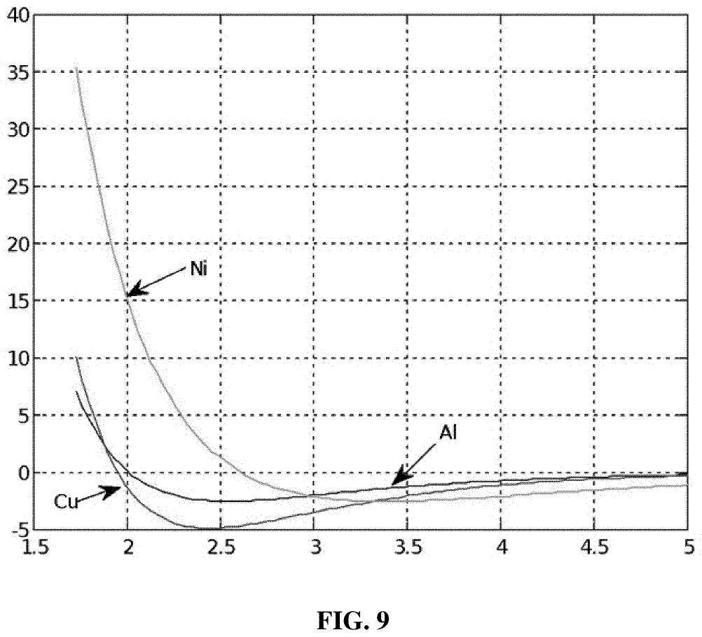

Calculating the dependence of the interaction forces between molecules on the distance using Formula (12) below can generate a graphical dependency as shown in FIG. 9:

$$F ( {r}_{0} ξ ) = {A}_{0} \frac{ξ - α}{{ξ}^{2}} {e}^{- β ξ} , \qquad ( 12 )$$

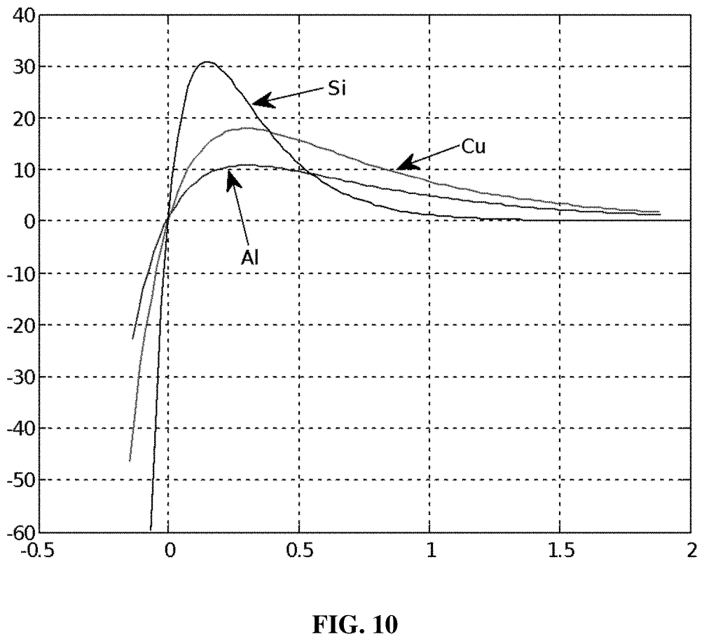

Calculating the nonlinear relationship between stresses and deformations of all-round tension-compression using Formula (4) below can generate a graphical dependency as shown in FIG. 10:

$$\frac{{S}_{0}}{N} {σ}^{*} ( e ) = \frac{2 π}{3} {\int }_{{r}_{n}}^{∞} F ( r ( 1 + e ) ) {r}^{2} dr \qquad ( 4 )$$

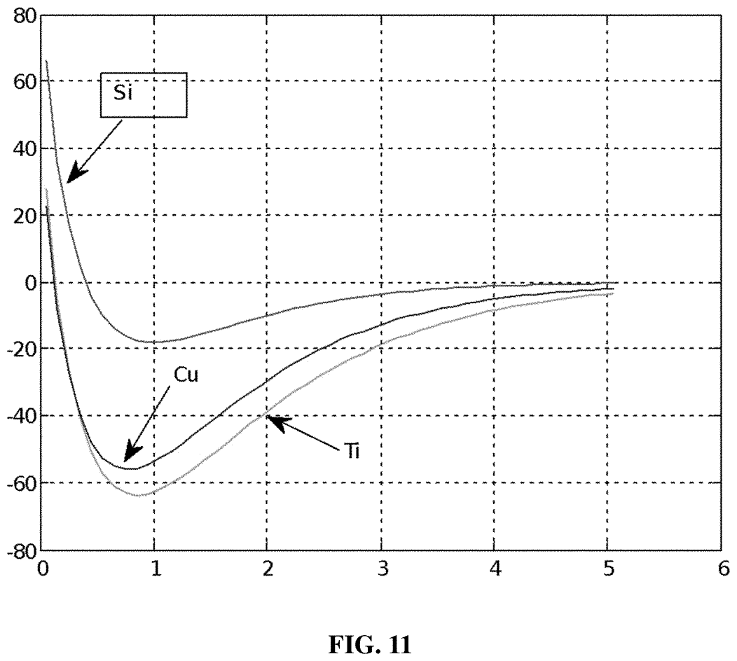

Calculating the dependence of the force of attraction of an external molecule to the surface of the body using Formula (17) below can generate a graphical dependency as shown in FIG. 11:

$${R}_{3} ( h ) = \frac{{V}_{0}}{3 {S}_{0}} {Φ}_{3} ( {r}_{0} h ) = 2 π {\int }_{0}^{π / 2} sin θ d θ {\int }_{{r}_{0}}^{∞} Φ ( z ) \frac{ξ cos θ + h}{z} {ξ}^{2} d ξ . \qquad ( 17 )$$

In step 310 the computing device 102 may transmit the resulting data 206 to the external node 190 via a network protocol, such as HTTP, to the IP address of the external node 190, as the IP address is stored. Next, in step 312, the external node adjusts operation using resulting data. In one embodiment the external node 190 is a physical vapor deposition vacuum process system used to deposit a very thin film onto a substrate, the system including a computer communicably connected to the communications network, the computer including a processor, a memory, and a programming module configured for receiving said resulting data from the computing device, over the communications network and adjusting a voltage, and a time of application of said voltage, in the physical vapor deposition vacuum process system according to said resulting data.

In another embodiment the external node 190 is a high-pressure, high-temperature press system used to produce an allotrope of an element, the system including a computer communicably connected to the communications network, the computer including a processor, a memory, and a programming module configured for receiving said resulting data from the computing device, over the communications network, and adjusting a temperature, pressure, and a time of application of said temperature and pressure, in the high-pressure, high-temperature press system according to said resulting data.

With regard to the step of calculating parameters

$$ξ = \frac{r}{{r}_{0}} ; α = \frac{{r}_{2}}{{r}_{0}} ; β = {br}_{0} ; {A}_{0} = \frac{A}{{r}_{0}}$$

of the specific molecule based on said initial data and said physical constraints, the following clarification is provided. The constants A, r₁, h of formula (11) above and the parameters α, β, B, of formula (12) above are calculated. The data for the calculation are found or looked up in a reference database (i database 104) of physical quantities. For example, for nickel:

$$volumetric heat capacity {C}_{p} = 4.09 * {10}^{6} \frac{J}{m 3 grad} ;$$

$$kinetic component of heat capacity {C}_{k} = 1.88 * {10}^{6} \frac{J}{m 3 grad} ;$$

$$coefficient of linear thermal expansion ϑ = 2.21 * {10}^{6} \frac{1}{grad} ;$$

$$bulk modulus K = 159 * {10}^{9} \frac{N}{{m}^{3}} ;$$

$$sublimation energy {W}_{s} = 64.8 * {10}^{9} \frac{J}{{m}^{3}} ;$$

$$the radius of the atom {r}_{0} = 149 * {10}^{- 12} m ;$$

$$body area per one molecule {S}_{0} = 4 {r}_{0}^{2} ;$$

$$the volume concentration of molecules is N = 9 * {10}^{28} \frac{1}{{m}^{3}} .$$

The parameters α, β, and B are calculated as follows. Parameters A₀ and α of formula (12) above are calculated using the following formulas:

$$α - 1 = \frac{3 θ + 1}{{β}^{2} θ + B + 3 θ β} ;$$

$${A}_{0} = \frac{3 {S}_{0} B}{2 π N {r}_{0}^{B}} ; B = \frac{{β}^{2} {σ}^{*} ( 0 )}{( 1 - β ( α - 1 ) ) {e}^{- β ξ}} ;$$

### Where:

$$θ = \frac{- C}{9 ϑ K } , C = ( {C}_{p} - {C}_{k} ) .$$

Parameter β is calculated using formula (15) above. The integral of formula (15) above has no analytical solution. The integral is calculated numerically for various values of β. The parameter β is chosen in such a way that equality (15) holds. For example, for nickel,

$$a = 2.74 , β = 0.65 , B = 344 * {10}^{9} \frac{N}{{m}^{2}} .$$

The constants A, r₁, b of formula (11) above are calculated as follows. Sing the tables above, parameters α, β, B are found using the following formulas:

$$A = \frac{3 {S}_{0} B}{2 π {Nr}_{0}^{s}} ; {r}_{1} = α {r}_{0} ; b = \frac{β}{{r}_{0}} .$$

FIG. 4 is a block diagram of a system including an example computing device 400 and other computing devices. Consistent with the embodiments described herein, the aforementioned actions performed by devices 102, 190 may be implemented in a computing device, such as the computing device 400 of FIG. 4. Any suitable combination of hardware, software, or firmware may be used to implement the computing device 400. The aforementioned system, device, and processors are examples and other systems, devices, and processors may comprise the aforementioned computing device. Furthermore, computing device 400 may comprise an operating environment for system 100 and process 300, as described above. Process 300 may operate in other environments and are not limited to computing device 400.

With reference to FIG. 4, a system consistent with an embodiment may include a plurality of computing devices, such as computing device 400. In a basic configuration, computing device 400 may include at least one processing unit 402 and a system memory 404. Depending on the configuration and type of computing device, system memory 404 may comprise, but is not limited to, volatile (e.g. random-access memory (RAM)), nonvolatile (e.g. read-only memory (ROM)), flash memory, or any combination or memory. System memory 404 may include operating system 405, and one or more programming modules 406. Operating system 405, for example, may be suitable for controlling computing device 400's operation. In one embodiment, programming modules 406 may include, for example, a program module 407 for executing the actions of devices 102, 190. Furthermore, embodiments may be practiced in conjunction with a graphics library, other operating systems, or any other application program and is not limited to any particular application or system. This basic configuration is illustrated in FIG. 4 by those components within a dashed line 420.

Computing device 400 may have additional features or functionality. For example, computing device 400 may also include additional data storage devices (removable and/or non-removable) such as, for example, magnetic disks, optical disks, or tape. Such additional storage is illustrated in FIG. 4 by a removable storage 409 and a non-removable storage 410. Computer storage media may include volatile and nonvolatile, removable and non-removable media implemented in any method or technology for storage of information, such as computer readable instructions, data structures, program modules, or other data. System memory 404, removable storage 409, and non-removable storage 410 are all computer storage media examples memory storage.) Computer storage media may include, but is not limited to, RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other medium which can be used to store information and which can be accessed by computing device 400. Any such computer storage media may be part of device 400. Computing device 400 may also have input device(s) 412 such as a keyboard, a mouse, a pen, a sound input device, a camera, a touch input device, etc. Output device(s) 414 such as a display, speakers, a printer, etc. may also be included. Computing device 400 may also include a vibration device capable of initiating a vibration in the device on command, such as a mechanical vibrator or a vibrating alert motor. The aforementioned devices are only examples, and other devices a ay be added or substituted.

Computing device 400 may also contain a network connection device 415 that may allow device 400 to communicate with other computing devices 418, such as over a network in a distributed computing environment, for example, an intranet or the Internet. Device 415 may be a wired or wireless network interface controller, a network interface card, a network interface device, a network adapter or a LAN adapter. Device 415 allows for a communication connection 416 for communicating with other computing devices 418. Communication connection 416 is one example of communication media. Communication media may typically be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media. The term computer readable media as used herein may include both computer storage media and communication media.

As stated above, a number of program modules and data files may be stored in system memory 404, including operating system 405. While executing on processing unit 402, programming modules 406 (e.g. program module 407) may perform processes including, for example, one or more of the stages of the process 300 as described above. The aforementioned processes are examples, and processing unit 402 may perform other processes. Other programming modules that may be used in accordance with embodiments herein may include electronic mail and contacts applications, word processing applications, spreadsheet applications, database applications, slide presentation applications, drawing or computer-aided application programs, etc.

Generally, consistent with embodiments herein, program modules may include routines, programs, components, data structures, and other types of structures that may perform particular tasks or that may implement particular abstract data types. Moreover, embodiments herein may be practiced with other computer system configurations, including hand-held devices, multiprocessor systems, microprocessor-based or programmable consumer electronics, minicomputers, mainframe computers, and the like. Embodiments herein may also be practiced in distributed computing environments where tasks are performed by remote processing devices that are linked through a communications network. In a distributed computing environment, program modules may be located in both local and remote memory storage devices.

Furthermore, embodiments herein may be practiced in an electrical circuit comprising discrete electronic elements, packaged or integrated electronic chips containing logic gates, a circuit utilizing a microprocessor, or on a single chip (such as a System on Chip) containing electronic elements or microprocessors. Embodiments herein may also be practiced using other technologies capable of performing logical operations such as, for example, AND, OR, and NOT, including but not limited to mechanical, optical, fluidic, and quantum technologies. In addition, embodiments herein may be practiced within a general purpose computer or in any other circuits or systems.

Embodiments herein, for example, are described above with reference to block diagrams and/or operational illustrations of methods, systems, and computer program products according to said embodiments. The functions/acts noted in the blocks may occur out of the order as shown in any flowchart. For example, two blocks shown in succession may in fact be executed substantially concurrently or the blocks may sometimes be executed in the reverse order, depending upon the functionality/acts involved.

While certain embodiments have been described, other embodiments may exist. Furthermore, although embodiments herein have been described as being associated with data stored in memory and other storage mediums, data can also be stored on or read from other types of computer-readable media, such as secondary storage devices, like hard disks, floppy disks, or a CD-ROM, or other forms of RAM or ROM. Further, the disclosed methods' stages may be modified in any manner, including by reordering stages and/or inserting or deleting stages, without departing from the claimed subject matter.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A system for calculating intermolecular forces, the system comprising:
a) a computing device communicably connected to a communications network, the computing device including a processor, a memory, an attached database, a user interface, a display and a programming module configured for:
1) reading initial data input by a user into the user interface, wherein said initial data includes an identity of a specific molecule;
2) reading physical constants corresponding to the specific molecule from the database, wherein said physical constants include elastic modulus, bulk elastic modulus, Poisson's ratio, heat capacity, thermal expansion coefficient, sublimation energy, heat of fusion, and volume change during melting;
3) calculating the following resulting data based on said initial data and said physical constraints: i) a non-linear relationship between stresses and deformation of a comprehensive tension-compression of the specific molecule, ii) an energy of sublimation of the specific molecule, iii) parameters
$$ξ = \frac{r}{{r}_{0}} ; α = \frac{{r}_{2}}{{r}_{0}} ; β = {br}_{0} ; {A}_{0} = \frac{A}{{r}_{0}}$$
of the specific molecule, iv) an interaction force between the specific molecule and an external surface of its body, v) a force acting on the specific molecule, wherein its displacement is relative to other molecules; and
4) transmitting said resulting data to an exterior node, over the communications network;
b) a physical vapor deposition vacuum process system used to deposit a very thin film onto a substrate, the system including a computer communicably connected to the communications network, the computer including a processor, a memory, and a programming module configured for:
1) receiving said resulting data from the computing device, over the communications network; and
2) adjusting a voltage, and a time of application of said voltage, in the physical vapor deposition vacuum process system according to said resulting data.

2. The system of claim 1, wherein the user interface further comprises a keyboard, wherein data input into the keyboard is presented on the display.

3. The system of claim 2, wherein the physical vapor deposition vacuum process system delivers the voltage across a low-pressure gas, thereby creating a high-energy plasma that enables the thin film to deposit on the substrate.

4. A system for calculating intermolecular forces, the system comprising:
a) a computing device communicably connected to a communications network, the computing device including a processor, a memory, an attached database, a user interface, a display and a programming module configured for:
1) reading initial data input by a user into the user interface, wherein said initial data includes an identity of a specific molecule;
2) reading physical constants corresponding to the specific molecule from the database, wherein said physical constants include elastic modulus, bulk elastic modulus, Poisson's ratio, heat capacity, thermal expansion coefficient, sublimation energy, heat of fusion, and volume change during melting;
3) calculating the following resulting data based on said initial data and said physical constraints: i) a non-linear relationship between stresses and deformation of a comprehensive tension-compression of the specific molecule, ii) an energy of sublimation of the specific molecule, iii) parameters
$$ξ = \frac{r}{{r}_{0}} ; α = \frac{{r}_{2}}{{r}_{0}} ; β = {br}_{0} ; {A}_{0} = \frac{A}{{r}_{0}}$$
of the specific molecule, iv) an interaction force between the specific molecule and an external surface of its body, v) a force acting on the specific molecule, wherein its displacement is relative to other molecules; and
4) transmitting said resulting data to an exterior node, over the communications network;
b) a high-pressure, high-temperature press system used to produce an allotrope of an element, the system including a computer communicably connected to the communications network, the computer including a processor, a memory, and a programming module configured for:
1) receiving said resulting data from the computing device, over the communications network; and
2) adjusting a temperature, pressure, and a time of application of said temperature and pressure, in the high-pressure, high-temperature press system according to said resulting data.

5. The system of claim 4, wherein the user interface further comprises a keyboard, wherein data input into the keyboard is presented on the display.

6. The system of claim 5, wherein the high-pressure, high-temperature press system is a press that produces a pressure of about 5 GPa at about 1500 degrees Celsius.
